(19) United States
(12) Patent Application Publication
(10) Pub. No.: US 2019/0159463 A1
(43) Pub. Date: May 30, 2019

(54) ENTEROBACTER CLOACAE BIOCONTROL STRAIN CAPABLE OF EFFECTIVELY INHIBITING ASPERGILLUS FLAVUS FROM SYNTHESIZING AFLATOXINS AND APPLICATION THEREOF

(71) Applicant: OIL CROPS RESEARCH INSTITUTE, CHINESE ACADAMY OF AGRICULTURAL SCIENCES, Wuhan (CN)

(72) Inventors: Peiwu LI, Wuhan (CN); Xiaoxia DING, Wuhan (CN); Qi ZHANG, Wuhan (CN)

(21) Appl. No.: 16/204,588

(22) Filed: Nov. 29, 2018

(30) Foreign Application Priority Data
Nov. 29, 2017 (CN) 201711230520.1

(51) Int. Cl. A01N 63/02; A01N 25/14; C12N 1/20; A23B 9/28

(52) U.S. Cl. CPC A01N 63/02; A01N 25/14; A23V 2002/00; A23B 9/28; C12N 1/20

(57) ABSTRACT

The present invention belongs to the field of microorganisms, and particularly relates to an Enterobacter cloacae biocontrol strain capable of effectively inhibiting Aspergillus flavus from producing aflatoxins and an application thereof. Enterobacter cloacae biocontrol strain 3J1EC was deposited in China Center for Type Culture Collection on Jun. 13, 2017, with the deposit address being Wuhan University, Wuhan, China, and the deposit number being CCTCC No. M 2017330. The strain can be used for inhibiting Aspergillus flavus from producing aflatoxins to prevent and control the contamination of food crops by aflatoxins.

### TECHNICAL FIELD

The present invention belongs to the field of microorganisms, and particularly relates to an Enterobacter cloacae biocontrol strain capable of effectively inhibiting production of aflatoxins by Aspergillus flavus and the application thereof.

### BACKGROUND

Aspergillus flavus is a pathogenic fungus capable of producing a class of highly carcinogenic and highly toxic mycotoxins, i.e., aflatoxins, including B, G and M groups, in which B1 is the most common and most toxic. It can widely contaminate food crops such as peanuts and corn, which seriously threatens the health of people and livestock and causes great economic losses. Therefore, it is imperative to strengthen the prevention and control of contamination by Aspergillus flavus and its toxins.

For the prevention and control of Aspergillus flavus, there are currently three kinds of prevention and control methods, i.e., physical, chemical and biological methods. However, chemical prevention and control methods are not only costly, but also easily pollute the environment; furthermore, the pathogenic fungus can easily generate tolerance or even resistance to chemical agents during the prevention and control of the pathogenic fungus. Physical prevention and control methods are time-consuming, labor-intensive, and not high in the rate of detoxification, and can easily cause loss of nutrients. However, biological methods have the advantages of safety, efficiency and durability. Therefore, the study on strengthening the biological prevention and control of Aspergillus flavus has important significance for the agricultural industry and economic benefits in China.

A variety of microorganisms can inhibit the production of aflatoxins, and studies have shown that Bacillus subtilis can significantly inhibit the infection of corncobs and the production of toxins by Aspergillus flavus; a fermentation broth of Bacillus pumilus can inhibit the production of aflatoxins; LIU, Shu et al. screened out a strain producing broad-spectrum, high-activity antimicrobial substance, i.e., actinomycete GB-2, from the Lianyungang sea area, which has a certain inhibitory effect on Aspergillus flavus, and isolated an actinomycete strain in the Turpan desert of Xinjiang, which produces an antibiotic that has an inhibitory effect on Aspergillus flavus; and KONG, Qing et al., from the School of Food Science and Engineering, Ocean University of China, found that a strain of marine Bacillus megaterium can inhibit production of aflatoxins, with an inhibition rate of about 87% in a culture medium, and can inhibit the biological synthesis of aflatoxins on mechanically damaged (punched) peanuts, whereas on peanuts without any mechanical damage, this Bacillus strain cannot inhibit toxin synthesis. In current research, there is no report on the inhibition of Aspergillus flavus by Enterobacter cloacae.

### SUMMARY OF THE INVENTION

The present invention provides an Enterobacter cloacae biocontrol strain 3J1EC capable of effectively inhibiting Aspergillus flavus from producing aflatoxins and the application thereof. The Enterobacter cloacae biocontrol strain 3J1EC in the present invention can significantly inhibit Aspergillus flavus from producing toxins, and has an excellent inhibitory effect on production of toxins by Aspergillus flavus in peanuts of various varieties and origins.

The Enterobacter cloacae biocontrol strain 3J1EC was deposited in China Center for Type Culture Collection (CCTCC) on Jun. 13, 2017, with the deposit address being Wuhan University, Wuhan, China, and the accession number being CCTCC No. M 2017330.

The Enterobacter cloacae biocontrol strain 3J1EC is obtained from the following method. A soil collected in a peanut field in Huangpi of Hubei was diluted with a gradient, and then 100 μL of the dilution was taken and spread over a plate of an LB solid culture medium, grown bacteria were picked up with an inoculating loop and transferred to a fresh LB solid medium for plate streaking, a single colony was picked up after such transfers several times, and subjected to a co-culture experiment in a culture medium with Aspergillus flavus, i.e., peanuts being ex vivo inoculated with Aspergillus flavus for a co-culture antimicrobial test, the toxin productivity was tested, and finally, a strain of 3J1EC, which has a significant effect on the toxin production of Aspergillus flavus, was screened out and deposited in China Center for Type Culture Collection (CCTCC) with an accession number being CCTCC No. M 2017330. The strain was identified by using a 16S rDNA specific amplification technique combined with morphological characteristics and physiological and biochemical experiments, and the results show that this is a strain of Enterobacter cloacae, belonging to the Enterobacteriaceae family and the Enterobacter genus.

TABLE 1
Main biological characteristics of Enterobacter cloacae 3J1EC:

| | | Ornithine | | | Lysine | | | |
|---|---|---|---|---|---|---|---|---|
| | | decar- | | Arginine | decar- | | | Culture |
| Gram | | boxylase | Indole | hydrolase | boxylase | | Culture | temper- |
| staining | Lactose | test | test | test | test | Glucose | time | ature |
| Negative | Positive | Positive | Negative | Positive | Negative | Positive | 12 h- | 25° C.- |
| | | | | | | | 24 h | 37° C. |

An inhibitor is provided for inhibiting Aspergillus flavus from producing aflatoxins. The inhibitor includes a fermentation product of the above-mentioned Enterobacter cloacae biocontrol strain 3J1EC.

In an embodiment, a preparation form of the inhibitor is a solid, a liquid or a powder.

In an embodiment, the inhibitor is a liquid, and the inhibitor has a final concentration of Enterobacter cloacae of (1-9)×10⁷ CFU/mL.

In an embodiment, the inhibitor is a fermentation broth of the Enterobacter cloacae biocontrol strain 3J1EC, and a final concentration of Enterobacter cloacae in the fermentation broth of the Enterobacter cloacae biocontrol strain 3J1EC is (1-9)×10⁷ CFU/mL. A method for preparing the fermentation broth of Enterobacter cloacae 3J1EC includes: activating Enterobacter cloacae 3J1EC on an LB plate, culturing the Enterobacter cloacae in an incubator at 25° C.−37° C. for 24 h, picking up a single colony of Enterobacter cloacae with a needle, transferring the single colony to a liquid medium, culturing with shaking for 12 h-24 h, pipetting and transferring 1%-3% of the culture liquid to a fresh liquid medium, and culturing with shaking for 12 h-24 h to obtain a fermentation broth of antagonistic Enterobacter cloacae 3J1EC.

An application of the above-mentioned inhibitor for inhibiting Aspergillus flavus from producing aflatoxins in inhibiting Aspergillus flavus from producing aflatoxins. The specific application method involves: coating a surface of a biological sample with the inhibitor for inhibiting Aspergillus flavus from producing aflatoxins or mixing the inhibitor with the biological sample to prevent and control the contamination of the biological sample by aflatoxins.

An application of the above-mentioned Enterobacter cloacae 3J1EC is provided for inhibiting Aspergillus flavus from producing aflatoxins. The specific application method includes: coating a surface of a biological sample with a fermentation broth of Enterobacter cloacae 3J1EC or mixing the inhibitor with the biological sample to prevent and control the contamination of the biological sample by aflatoxins.

The present invention has the following beneficial effects:

the present invention isolates a strain of Enterobacter cloacae 3J1EC for the first time, which has a better prevention and control effect against Aspergillus flavus, from the soil. The strain can be used for inhibiting Aspergillus flavus from producing aflatoxins, preventing and controlling contamination of food crops by aflatoxins.

### DETAILED DESCRIPTION

In the invention, a soil collected in a peanut field in Huangpi of Hubei was diluted with a gradient, and then 100 μL of the dilution was taken and spread over a plate of an LB solid culture medium for culture, grown bacteria were picked up with an inoculating loop and streaked onto a fresh LB solid medium plate, a single colony was picked up after performing such plate streaking for several times and co-cultured in a culture medium with Aspergillus flavus, and was then tested for the toxin productivity, peanuts were ex vivo inoculated with Aspergillus flavus for a co-culture antimicrobial test, and a strain of 3J1EC, which has a significant effect on the toxin production of Aspergillus flavus, was screened out and deposited in China Center for Type Culture Collection (CCTCC), with an accession number being CCTCC No. M 2017330.

### Example 1

1) Enterobacter cloacae 3J1EC was activated on an LB plate, and cultured in an incubator at 37° C. for 24 hours, and a single colony of activated Enterobacter cloacae was picked up with a needle, and the single colony was transferred to a conical flask containing 15 mL of LB liquid medium, and cultured at 28° C. with shaking at 200 r·min-1 for 12 hours. 1% of the culture liquid was pipetted and transferred to a conical flask containing 15 mL of LB liquid culture medium, and cultured at 28° C. with shaking at 200 r·min-1 for 12 hours to obtain a fermentation broth of the antagonistic strain.

2) The fermentation broth of Enterobacter cloacae (with a final concentration of 1×10⁷ CFU/mL) was co-cultured in a Sabouraud's liquid culture medium together with a suspension of vigorously grown Aspergillus flavus (with a final spore concentration of 5.0×10⁵ spores·mL⁻¹), which was cultured for 7 days. The co-culture was carried out at 28° C. and 200 rpm for 5 days. 3 replicates were set for each process.

3) The content of aflatoxin B1 in the culture liquid was measured (Table 2).

TABLE 2
Prevention and control effect of the biocontrol
bacteria against Aspergillus flavus

| | | A. flavus + CCTCC M |
|---|---|---|
| Treatment | A. flavus | 2017330 |
| AFB1 toxin content (ng/ml) | 215 ± 36.67 | 11.2 ± 1.79 |

It can be seen from the above experimental results that the inhibition rate of Enterobacter cloacae CCTCC No. M 2017330 against the aflatoxin is about 94.8%, indicating that it has the ability to inhibit the production of aflatoxin.

### Example 2

1) Peanuts kernels of Zhonghua No. 6 were taken from a peanut field in Hubei and ground into powder, 1 g of the peanut powder was weighed into a culture dish, and 1 ml of an Aspergillus flavus spore solution (5×10⁵ spores/mL) and 1 ml of an Enterobacter cloacae 3J1EC (CCTCC M 2017330) solution (1×10⁷ CFU/mL) were simultaneously added thereto; in addition, a control was provided by replacing the CCTCC M 2017330 solution with a Sabourand culture medium;

2) the inoculated peanut powder was cultured in an incubator at 28° C. for 9 days, 15 mL of 70% aqueous methanol was added thereto, and the mixture was vortexed and then placed on a shaker for 30 min. 3 mL of a supernatant was taken, 8 mL of ultrapure water was added thereto, and vortex centrifugation was carried out; and

3) 8 mL of a supernatant was taken and tested by using an immunoaffinity column-HPLC method for the content of aflatoxin B1 (Table 3), with three replicates being set in the test.

TABLE 3
Prevention and control effect of the biocontrol
bacteria against Aspergillus flavus

| | | A. flavus + |
|---|---|---|
| Treatment | A. flavus | CCTCC M 2017330 |
| AFB1 toxin content (ng/ml) | 516.61 ± 51.91 | 78.27 ± 2.98 |

It can be seen from the above experimental results that the inhibition rate of the strain of CCTCC M 2017330 on Aspergillus flavus from producing toxins on the peanuts of Zhonghua No. 6 is about 85%, indicating that the strain has a good prevention and control effect against aflatoxins during the storage of peanuts.

### Example 3

1) 10 peanut kernels of Luhua No. 8 were taken from a peanut field in Anhui, the surface of the peanuts was coated with a fermentation broth of Enterobacter cloacae 3J1EC, while 1 ml of Aspergillus flavus spore solution (5×10⁵ spores/mL) was added thereto; in addition, a control was provided by replacing the fermentation broth of CCTCC M 2017330 with a Sabourand culture medium;

2) the inoculated peanut kernels were cultured in an incubator at 28° C. for 9 days. Thereafter, the peanut kernels were ground into a peanut powder, and added with 15 mL of 70% aqueous methanol to obtain a mixture. The mixture was vortexed and then placed on a shaker for 30 min.

3 mL of a supernatant was taken, 8 mL of ultrapure water was added thereto, and vortex centrifugation was carried out; and

3) 8 mL of a supernatant was taken and tested by using an immunoaffinity column-HPLC method for the content of aflatoxin B1 (Table 4), with three replicates being set in the test.

TABLE 4
Prevention and control effect of the biocontrol bacteria
against Aspergillus flavus in peanuts

| Treatment | A. flavus | A. flavus + CCTCC M 2017330 |
|---|---|---|
| Toxin content (ng/ml) | 449.95 ± 42.51 | 58.23 ± 2.23 |

It can be seen from the above experimental results that the inhibition rate of the strain of CCTCC M 2017330 on Aspergillus flavus from producing toxins on the peanuts of Luhua No. 8 is about 87%, indicating that the strain also has a good prevention and control effect for peanuts of different varieties.

1. An inhibitor for inhibiting production of aflatoxins by Aspergillus flavus, comprising a fermentation product of an Enterobacter cloacae biocontrol strain 3J1EC, and the Enterobacter cloacae biocontrol strain 3J1EC is deposited in China Center for Type Culture Collection (CCTCC) on Jun. 13, 2017, with an accession number being CCTCC No. M 2017330.

2. The inhibitor according to claim 1, wherein the inhibitor is a liquid, a spray powder, a dry wettable powder, or dry wettable granules.

3. The inhibitor according to claim 1, wherein the inhibitor is a liquid, and a final concentration of Enterobacter cloacae in the inhibitor is (1-9)×10⁷ CFU/mL.

4. The inhibitor according to claim 3, wherein the inhibitor is a fermentation broth of Enterobacter cloacae 3J1EC, a final concentration of Enterobacter cloacae in the fermentation broth of Enterobacter cloacae 3J1EC is (1-9)×10⁷ CFU/mL, and a method for preparing the fermentation broth of Enterobacter cloacae 3J1EC includes: activating Enterobacter cloacae 3J1EC on an LB plate, culturing the Enterobacter cloacae in an incubator at 25° C.-37° C. for 24 h, picking up a single colony of Enterobacter cloacae with a needle, transferring the single colony to a liquid medium, culturing with shaking for 12 h-24 h, pipetting and transferring 1%-3% of culture liquid to a fresh liquid medium, and culturing with shaking for 12 h-24 h to obtain an antagonistic fermentation broth of strain Enterobacter cloacae 3J1EC.

5. A method for inhibiting Aspergillus flavus from producing aflatoxins, comprising: coating a surface of a biological sample with an inhibitor of claim 1 or mixing an inhibitor of claim 1 with a biological sample to inhibit Aspergillus flavus from producing aflatoxins.

6. A method for preparing the fermentation broth of Enterobacter cloacae 3J1EC comprising:
activating Enterobacter cloacae 3J1EC on an LB plate,
culturing the Enterobacter cloacae in an incubator at 25° C.−37° C. for 24 h,
picking up a single colony of Enterobacter cloacae with a needle,
transferring the single colony to a liquid medium,
culturing with shaking for 12 h-24 h, pipetting and transferring 1%-3% of culture liquid to a fresh liquid medium, and
culturing with shaking for 12 h-24 h to obtain an antagonistic fermentation broth of strain Enterobacter cloacae 3J1EC,
wherein the Enterobacter cloacae biocontrol strain 3J1EC is deposited in China Center for Type Culture Collection (CCTCC) with an accession number being CCTCC No. M 2017330
